(19) United States
(12) Patent Application Publication
(10) Pub. No.: US 2015/0182586 A1
(43) Pub. Date: Jul. 2, 2015

(54) DAIRY FARM TEAT DIP COMPOSITIONS AND METHODS

(71) Applicant: Advanced BioCatalytics Corporation, Irvine, CA (US)

(72) Inventors: John W. BALDRIDGE, Irivne, CA (US); Carl W. PODELLA, Irvine, CA (US); Andrew H. MICHALOW, Irvine, CA (US); Michael G. GOLDFELD, Irvine, CA (US); Kevin JOHANSEN, Irvine, CA (US); Andrew D. MALEC, Irvine, CA (US)

(21) Appl. No.: 14/587,541

(22) Filed: Dec. 31, 2014

Related U.S. Application Data
(60) Provisional application No. 61/922,727, filed on Dec. 31, 2013.

(51) Int. Cl. A61K 38/16; A61K 31/19; A61K 33/40

(52) U.S. Cl. CPC A61K 38/16; A61K 33/40; A61K 31/19

(57) ABSTRACT

Disclosed herein are teat dip compositions, comprising a) a fermentation derived mixture that include low molecular weight protein component; b) an emollient; c) one or more surfactants; and d) an anti-microbial agent. The disclosed methods provide for preventing milk contamination and associated bacterial infections of the teat in dairy animals, the method comprises applying to the teat of the dairy animal a composition, comprising: a) a fermentation derived mixture that include low molecular weight protein component; b) an emollient; c) one or more surfactants; and d) an anti-microbial agent, wherein the application is effective in treating mastitis of the teat, and wherein the composition is gentle on the teat tissue.

### RELATED APPLICATIONS

This application claims the benefit of U.S. Provisional Application No. 61/922,727, filed Dec. 31, 2013, by John W. BALDRIDGE, et al., and entitled “DAIRY FARM TEAT DIP COMPOSITIONS AND METHODS,” which is incorporated herein by reference in its entirety.

### FIELD OF THE DISCLOSURE

The present disclosure pertains to the field of dairy animal maintenance, in particular compositions of teat dips, and their methods of use for the prevention of milk contamination and bacterial infections such as those causing mastitis in dairy animals, by cleaning and disinfecting with a composition that is gentle on the teat tissue in industrial milking operations. The compositions may comprise (a) a germicidal agent, (b) surfactants, (c) proteins derived from heat stressed yeast and optionally, and may include emollients, moisturizers, pH buffers and colorants.

### BACKGROUND OF THE DISCLOSURE

Mastitis is an inflammatory condition of the mammary glands, and/or the adjacent tissue in the udder of dairy animals. It may be caused by bacterial infection and is the most costly disease in the dairy industry, based on lost production. Estimates of the costs to industry vary. However, in the United States alone, these costs are reportedly over $2 billion per year. Losses can range from a significant reduction in the milk yield of the producing animals, to stoppage of production. In extreme cases, mastitis can result in the death of the animal. Quality of the milk can be compromised, as well.

There are many root causes of mastitis. Dairy cows are continuously exposed to contaminants and pathogens both before and after the milking process. Further, in the dairy industry, there is a desire to maximize the duration of machine milking, while at the same time minimizing irritation or damage to teat tissue and udder. The damage to udder tissue by machine milking may be followed by the exposure to microbial pathogens resulting in mastitis. Maintaining a healthy teat tissue condition is an extremely important factor in milking operations. The udder and teats of an infected cow may be treated with an antibiotic, however the milk from such a cow cannot be sold until the antibiotic is reliably removed, which can take about five days after the last treatment.

A key approach to prevent the spread of mastitis is to use a germicidal “teat dip.” Normal practice is to treat the cow teats with an antimicrobial teat dip, either by dipping or spraying, before and after milking. Teat dips can be broken down into two distinct classes: non-barrier and barrier type. Non-barrier teat dips have traditionally focused on the use of fast-acting anti-microbial compositions for both pre-milking and post-milking operations. Barrier type teat dips, used mostly in the post-milking operation, typically also comprise an antimicrobial agent, but are applied for longer term contact and form a coating or a film protecting the teat skin from microbes that otherwise would have access and infect the skin.

Though there are numerous treatments available to prevent and treat mastitis, the industry losses indicate that there is a continued need to improve treatments to prevent and control the disease. For example, an iodine based products may be used for both pre- and post-milking as 0.5% Iodine pre-milk and 1% Iodine post-milk applications. Historically, two to three new mastitis cases developed every week with this teat dip, in a dairy farm milking about 150 cows.

Faster acting contaminant removal has not generally been a focus of teat dips. There is need for improved teat dip compositions and their methods of use for the prevention of milk contamination and bacterial infections such as those causing mastitis in dairy animals. The disclosed compositions and methods focus on the ability of the protein-surfactant based compositions to act in such a manner.

### SUMMARY OF THE DISCLOSURE

In one embodiment, the disclosed teat dip composition comprises: a) a fermentation derived mixture that include low molecular weight protein component; b) an emollient; c) one or more surfactants; and d) an anti-microbial agent.

In another embodiment, the disclosed methods provide for preventing milk contamination and associated bacterial infections of the teat in dairy animals, the method comprises applying to the teat of the dairy animal a composition, comprising: a) a fermentation derived mixture that include low molecular weight protein component; b) an emollient; c) one or more surfactants; and d) an anti-microbial agent, wherein the application is effective in treating mastitis of the teat, and wherein the composition is gentle on the teat tissue.

### DETAILED DESCRIPTION OF THE EMBODIMENTS

It is generally understood that exposing the teats to an effective teat dip solution before and after milking is the single most important procedure for reducing mastitis infections. Factors that contribute to the onset of mastitis are discussed.

Streptococcus agalactiae and Staphylococcus aureus bacteria account for the vast majority of all mastitis cases. Symptoms may include inflammation of the teat, which leads to reduced yield of milk. Furthermore, this can cause the teat to remain unsealed after milking, which leaves an open path for the pathogens to travel into the teat canal, and finally into the udder. Good teat health requires not only good hygiene practices, but proper handling procedures of dairy animals. The most fundamental requirements are to keep the teat surface free of pathogens and to minimize irritating factors, which includes the application of chemicals, mechanical handling and environmental conditions. Also, cuts and abrasions are additional sites where pathogens can penetrate and grow.

Disinfecting the teat surface has traditionally been the primary method to prevent propagation of disease. However, removing contaminants by so-called udder wash solutions is also important in the pre-milking step. Some teat dips act as both disinfectants for pre-milking, and for post-milking treatment. Traditionally, the main purpose of the teat dip is to kill and reduce the number of pathogens on the teat surface, to prevent them from spreading, which would otherwise compromise milk production, quality and yield.

Since disinfecting agents can sensitize the skin, their use has to be balanced against the antagonistic effects they might have on skin. Emollients or moisturizers are added to provide a soothing effect on the teats, and in some cases include glycerol or propylene glycol. Healthy teat skin has a pink hue and is soft, being generally free of lesions, sores scabs and calluses.

While killing pathogens is important in controlling mastitis, pathogens can harbor themselves in organic and inorganic contaminants. Contaminants can antagonize skin, and removing them provides a condition more conducive to healthy skin. Surfactants are important cleaning components, but some types of surfactants can be antagonistic to skin. Surfactants that are gentle on the skin are in some cases less effective in cleaning. Maintaining healthy teat skin helps the bovine immune system fight infection. Emollients are added to teat dip formulations to improve the skin condition.

A number of different types of disinfectants are used in teat dips, including iodine with iodophors, chlorhexidine, acidified sodium chlorite, organic acids, peroxides, quaternary ammonium chlorides and others. Iodophors have been the most widely used so far. Chlorhexidine and chlorite based products are more difficult to work with and are hazardous to the user, so handling of the product becomes critical to maintain a level of safety for workers. Due to regulatory restrictions and supply issues, there is also a need to move away from iodine based teat dips. Iodophors can also impart a taste to the milk if residues accidentally contaminate the milk, perhaps due to improper wiping after the pre milking dip procedure.

Whichever disinfectant is used, its ability to kill requires direct contact with the pathogen. Further, the disinfectant has a limited time where it is retained on the treated surfaces, which means that it has to have a fast kill rate to be effective. The more effective the kill, the less likely that there will be a critical propagation of the pathogen once the disinfectant is wiped off in pre-milking or sloughed off in a post-milking application. The downside to a strong disinfectant is that it can create an inflammatory condition in the teat surface, which makes it more prone to microbial attack. While emollients can soothe the skin somewhat, they can also inhibit disinfectant performance, so maintaining the proper balance under a broad range of conditions is a major challenge.

Iodophors are iodine based disinfectants and the antimicrobial active is free iodine, a well-known oxidizing agent. Since iodine per se is practically insoluble in water, formulating effective iodine-based teat dips to cover the wide range of environmental conditions is challenging. To provide a fast kill rate, a relatively high level of free iodine has to be maintained, and this causes sensitivity issues with the teat skin. The addition of emollients can soothe the teat skin, but the emollients can sometimes interfere with the ability of the free iodine to contact all the pathogens, impeding its antimicrobial effectiveness. Gradle (see U.S. Pat. No. 7,153,527) teaches and discloses using propylene glycol emollient at a level of between 50% to 99%, with an iodine complex.

Another group of active compounds that are gaining favor are organic acids. These include, but are not limited to: lactic acid, dodecylbenzene sulfonic (DDBSA), caprylic, salicylic, glycolic and capric acids. Lactic acid and DDBSA have generated the most data for teat dip applications (6). Lactic acid is a widely used ingredient in skin care products, where it serves to adjust pH (to moderately acidic values), as a humectant and a mild keratolytic agent. It is a descaler, soap-scum remover, and a registered anti-bacterial agent. Its application is also part of the trend toward environmentally safer and natural ingredients. The same is true for the capric and caprilic acids which are especially attractive in this context since they are naturally present in milk. DDBSA is a strong acid and displays a better pronounced antimicrobial effect, but it has been shown to be irritating to the skin. This means that a higher level of emollients and skin conditioning agents are required. DDBSA's use is typically recommended as being limited to use for pre-milking where exposure with skin is limited.

### Mastitis and Biofilms

One major phenomenon that weakens the ability of antimicrobials to be their most effective is the formation of biofilms at the teat surface. Biofilms have been known to reduce the ability of antimicrobial and antibiotic agents to come into contact with targeted pathogens in many areas. Specific to cow teat treatment, a number of studies cite the effects of biofilm formation as a significant complicating factor in mastitis treatment with teat dips. It is hypothesized that a key reason for the high level of mastitis losses are due to biofilms. The biofilm protects pathogens from the disinfectants and prevents their destruction. (2, 4 and 5). Furthermore, “[r]ecurrent infections are often attributable to biofilm growth of bacteria”. (2).

“Some Staphylococcus aureus strains, identified as causative agents of mastitis in cattle, exhibit the ability of producing a viscous extracellular polysaccharide layer, or slime, which is a biofilm. Today, it is considered to be a virulence factor, as it promotes bacterial adhesion onto the mammary epithelial cells and protects bacteria from opsonization and phagocytosis.” (4). “Research indicates the cure rate for Staph. aureus infected cows after treatment ranges from 4% to 92%, with overall success averaging a dismal 20% to 30%.” (5).

There are several deficiencies in the use of current antimicrobial compositions as teat dips. They rely on the use of antimicrobials at levels that irritate and can cause inflammation of the teat, which then necessitate high levels of emollients to soothe their negative effects. Further, they do not address the issue of penetrating through biofilms that harbor the pathogens, which subsequently prevent the control of persistent mastitis. The protein-enhanced compositions of the present disclosure has shown that they can be effective in penetrating biofilms, and presumably would be more efficient in helping to eliminate them as contaminants from the bovine teat surfaces.

It would be an advantage for the dairy industry to have a teat dip composition that could both remove pathogens that cause mastitis with minimal irritation of the teat skin to help preserve the health of the teat skin. Contented cows yield more milk, as there is an inverse relationship between irritation and productivity. Attacking pathogens by merely focusing on a teat dip's antimicrobial activity limits its effectiveness. Since pathogens are acquired with and harbored by contaminants on the skin, improved antimicrobial activity can be accomplished by removing contamination from the skin surface. Cleaning starts with comprehensive wetting and spreading of the teat dip composition over the surface to be cleaned, including penetration into folds and cracks of the skin. Therefore it is highly desirable to develop teat dip compositions with improved cleansing properties that are gentle on the skin. A less irritating teat dip would also reduce the level of emollients that are needed. Lower emollient levels could reduce pathogen growth rates by allowing post-milking teats to dry more quickly, and would improve effectiveness of any antimicrobial that would be used by reducing their action as a type of barrier. It is desirable, however, to have a teat dip composition where the post milking dip would remain on the skin surface after milking long enough for the teat to close, to prevent pathogens from entering the teat canal and udder.

An additional desired feature for a teat dip would be for it to penetrate biofilms that form on the teat skin, thereby more effectively removing them from the teat surface. Furthermore, there is a need to provide a type of “barrier,” or residual film that can control biofilm propagation, which would translate in persistent cases of mastitis being controlled. A teat dip that is effective in not only killing residual pathogens, but in removing them, including those tied up in a biofilm and soil, from the surface of the teat, would reduce the amount of antimicrobial agent needed and benefit the cow's teat condition. Removal of the pathogen through cleaning serves the same purpose as killing it. It is also desirable that the antimicrobial agent remains on the teat dip in a post milking step in an amount to protect against any residual pathogens. It is desirable to have a teat dip that employs an effective level of antimicrobial agent that still protects the cow, in order to reduce the negative, inflammatory effects of antimicrobials on the teat skin and improve the efficiency of the milking process, while minimizing attack by mastitis producing pathogens. Finally, it is desirable to have a teat dip composition that can be produced in a concentrate form, to minimize shipping costs, and dilute the teat dip on site, or close to the end use.

### Milk Quality

One of the key ways that a dairy can quantitatively determine milk quality is by measuring the somatic cell count (SCC) in the milk. SCC is also an indicator of a cow's health. Somatic cell count acts as an indicator of a cow's susceptibility to mastitis, and when the entire herd is monitored, it quantifies the effectiveness of the dairy's hygiene practices.

Somatic cells are mainly white blood cells (leukocytes) that entered the mammary gland as an immune response due to infection or injury. SCC can also include epithelial cells, which are milk producing cells that are shed from the lining of the udder as a result of an infection. Finally, any dead cells that may slough off of the teat during milking can contaminate the milk. Because they can harbor pathogens, they can cause temporary increases in SCC until the health of the teat improves.

SCC is measured as the number of cells in a milliliter of milk. A level under 100,000 indicates that the cow is not infected. In some embodiments, the disclosed teat dip compositions and methods of application result in treated cows having a measured SCC under 1000,000. At levels above 200,000, the likelihood that a cow will become infected with mastitis increases significantly to at least once per quarter. A level above 400,000 is considered as milk unfit for human consumption by the European Union. Regulatory limits in the USA go up to 750,000, but many customers pay premiums for lower SCC's. Production of cheese and yogurt requires lower SCC's, as well. Another important factor regarding SCC is that the price paid for milk that has a low SCC has a higher price than one with a higher SCC.

SCC is also used as a monitoring tool to track the health of a milking herd of cows. When SCC levels increase, then the chances for developing clinical mastitis increases. Clinical mastitis is when a milking cow's milk is isolated while the cow undergoes antibiotic treatment to bring the cow back to health. A further negative side effect of developing mastitis is that a cow that has had mastitis will generally provide lower milk production than what it was generating prior to developing mastitis. The severity of the mastitis determines how future milk yields are affected for a particular cow.

As discussed herein, the protein enhanced surfactant based teat dip compositions can be effective in treating mastitis of the teat while being gentle on the teat tissue. This could increase the effectiveness of a dairy's hygiene practices.

The disclosed teat dip composition comprises: a) a fermentation derived mixture that include low molecular weight protein component; b) an emollient; c) one or more surfactants; and d) an anti-microbial agent.

The compositions disclosed herein comprise exo-proteins, a surfactant or mixture of surfactants, emollients and an antimicrobial agent (e.g., hydrogen peroxide). Hydrogen peroxide has been employed due to its effectiveness as an antimicrobial agent, safety, benign residual effects, minimal antagonistic effects on teat skin and the absence of side-products after its application.

The disclosed methods provide for preventing milk contamination and associated bacterial infections of the teat in dairy animals, the method comprises applying to the teat of the dairy animal a composition, comprising: a) a fermentation derived mixture that include low molecular weight protein component; b) an emollient; c) one or more surfactants; and d) an anti-microbial agent, wherein the application is effective in treating mastitis of the teat, and wherein the composition is gentle on the teat tissue.

In some embodiments, the method improves the rate of cleaning and contaminant removal. In some embodiments, the method removes excess dead skin cells from the teat and udder skin. In some embodiments, the method breaks down, removes and prevents biofilms that could harbor mastitis causing bacteria or other pathogens. In some embodiments, the method is performed for both pre and post milking, and during a 24 day treatment the number of cases of diary animals with clinical mastitis is reduced.

Rapid bacterial kill is an essential feature for teat dips, especially in the pre-milking step that usually provides about one minute exposure time. In some embodiments, the method has an application that is performed with dipping or spraying. In some embodiments, the method has an application time less than about 1 minute.

### Yeast Extracts

In some embodiments, the protein component comprises a fermentation broth recovered from a yeast fermentation process. In some embodiments, the protein component comprises a mixture of multiple intracellular proteins, or at least a portion of the mixture including yeast polypeptides obtained from fermenting yeast and yeast heat shock proteins resulting from subjecting a mixture obtained from the yeast fermentation to heat shock. In some embodiments, the yeast is selected from the group consisting of, but not limited to, Saccharomyces cerevisiae, Kluyveromyces marxianus, Kluyveromyces lactis, Candida utilis, Zygosaccharomyces, Pichia pastoris, and Hansanula polymorpha.

Yeast extracts disclosed herein, containing living yeast exo-proteins, were developed to take advantage of a synergy that was found between certain nonenzymatic yeast exo-proteins and surfactants. Moreover, the disclosed yeast exo-proteins show stability in a wide range of oxidizing and chemical conditions. In some embodiments, the concentration of the protein component in the composition of between about 1% and 60%, 1% and 50%, 10% and 50%, or 20% and 50%. In some embodiments, the composition is a diluted ready to use form that has a protein component concentration of less than 20%, 15%, 10% or 5%.

Yeast stress proteins included in the present disclosure include those released into the external solution in a fermentation process in response to various stress conditions, such as heat shock, starvation, radiation, chemical, mechanical stress, or combinations thereof. Stress proteins are formed and released into the medium by living cells due to the stress-induced expression of certain genes encoding these exo-proteins.

It has been shown that certain fermentation-derived stress-induced exo-proteins act as surfactant synergists. (U.S. Pat. Nos. 8,188,028; 7,645,730; and 7,659,237). The yeast exo-proteins, when combined with surfactants, show improved performance as compared to the surfactants alone in the wetting and spreading of aqueous solutions, including on living skin and compositions containing bioactives. (see U.S. patent application Ser. No. 14/279,352, now published as US 2014/0248373 and U.S. patent application Ser. No. 13/850,931, now published as US 2013/0251660).

Yeast cells release a certain amount of exo-proteins into the external solution in a regular fermentation process and a set of stress proteins in response to various stress conditions, such as heat shock, starvation, radiation, chemical, mechanical stress, or combinations thereof. Stress proteins are formed and released into the medium by living cells due to the stress-induced expression of certain genes encoding these exo-proteins.

In particular, heat has been shown to be a reliable and reproducible source of stress for yeast exo-protein production. Their processes and methods take advantage of proteins derived from yeast fermentation, including heat shock proteins. (see U.S. Pat. Nos. 7,476,529; 7,645,730; 7,659,237; and 7,759,301; and U.S. patent application Ser. No. 14/279,352, now published as US 2014/0248373). The entire disclosure of the above-referenced patent applications and patents, in particular the discussion on the production of stress proteins (for example, column 3, 1. 41 to column 4, 1. 51 of U.S. Pat. No. 7,659,237) are incorporated by reference herein.

In some embodiments, further comprising refining the aerobic fermentation supernatant and retaining those peptides having a molecular weight a mixture of proteins that include proteins having a molecular weight of between about 100 and about 450,000 daltons, the mixture of proteins being obtained from the fermentation of yeast and comprising a fermentation broth recovered from a yeast fermentation process. In some embodiments, further comprising refining the aerobic fermentation supernatant and retaining those peptides having a molecular weight of less than about 30,000 daltons. In some embodiments, further comprising refining the aerobic fermentation supernatant and retaining those peptides having a molecular weight of less than about 24,000 daltons. In some embodiments, further comprising refining the aerobic fermentation supernatant and retaining those peptides having a molecular weight of less than about 17,000 daltons. In some embodiments, further comprising refining the aerobic fermentation supernatant and retaining those peptides having a molecular weight of between about 6,000 daltons and about 17,000 daltons.

“Heat shock proteins”, or “stress proteins” (1), define a particular sub-set of the exo-protein component of the present disclosure, display properties related to the following, when combined with a surfactant. These properties are understood to be the basis in-part for the improvements in the teat dip composition, as follows:

(a) improving surfactant performance in terms of lowering interfacial tension, surface tension, and critical micelle concentration, spreading and wetting of the skin surface.

(b) complexes of surfactants with yeast stress proteins have been shown to enhance the antimicrobial efficiency of hydrogen peroxide, increasing the killing rate of certain bacteria. (see U.S. patent application Ser. No. 14/279,352, now published as US 2014/0248373). Such an enhancement allows for reducing the concentration of the antimicrobial active, such as hydrogen peroxide, thus also reducing the skin irritation, while retaining the sanitizing effect. The addition of emollients does not exhibit signs of inhibiting the antimicrobial activity with the teat dip compositions herein.

(c) complexes of surfactants with yeast stress proteins accelerate primarily aerobic microbial metabolic rates with a mechanism shown to rely, at least partially, on the uncoupling of oxidative phosphorylation in bacterial cells. The primary benefit of this feature is the ability to control biofilms by directing microbial metabolic processes towards ultimate oxidation of nutrient up to carbon dioxide, instead of biosynthesis and processes that depend on it, such as proliferation and/or generation of exocellular material used in the formation of biofilms.

It has been shown that the fermentation derived exo-proteins improve wetting on human skin. (see U.S. patent application Ser. No. 13/850,931, now published as US 2013/0251660). The net effect of the lowered interfacial tension, thereby translates into improved wetting of the skin surface, results in an accelerated removal of contamination off of cow's teat skin in the pre-milking cleaning stage. This is analogous to the wetting and cleaning of human skin. In addition, the teat dip composition of the current disclosure then also has benefits for post-milking application.

The exo-proteins enhance surface activity of synthetic and bio-derived surfactants, as revealed by the reduced oil/water interfacial tension, surface tension and critical micelle concentration of surfactant solutions. Surfactant activity is an important property in a teat dip, both because it facilitates the access of germicidal materials into the cracks and folds of the skin and because the dip also works as a washing agent, eliminating dirt and bacterial contaminations from the udder skin. Proteins used in the abovementioned applications are Saccharomyces Cerevisiae yeast stress exo-proteins (i.e., the proteins released by the living yeast cells in response to a stress, such as a mild, non-lethal heat shock). Their preparation does not require any disruption of yeast cells (mechanical or otherwise), and the material thus produced does not contain yeast cells, or any other living cells, or cell debris, or biochemical species coming from the interior of the yeast cells. The improved wetting of skin using the exo-protein and surfactant combination has been demonstrated. (see U.S. patent application Ser. No. 13/850,931, now published as US 2013/0251660). Improved wetting of the skin, including teat skin, enhances the speed and effectiveness of removing contaminants off of surfaces.

The exo-proteins were also shown to remove biofilms and preventing their formation in various settings, which may constitute a substantial advantage in confronting bacterial contaminations at the surface of teat and udder skin. Removing biofilms is helped by the regular application of the protein based compositions and methods of the present disclosure, where cows are milked twice per day, and sometimes three, with teat dip application during each milking cycle. In some embodiments, the teat dip composition is applied once, twice or three times a day.

### Germicidal Agent

Hydrogen peroxide, as a sanitizing agent, has the inherent benefit of breaking down into water and oxygen leaving no residues or side products. It has been used as a teat dip additive (6) and has shown to possess good antimicrobial properties over a broad spectrum of pathogens. Furthermore, hydrogen peroxide obviates the issue of a bacterial mutagenesis to combat antimicrobial Hydrogen peroxide has also shown compatibility and efficacy improvements in conjunction with the exo-proteins. (see U.S. patent application Ser. No. 12/581,007, now published as US 2010/0099599). Hydrogen peroxide is often combined with organic acids as an effective teat dip antimicrobial combination. Hydrogen peroxide is a minimal skin irritant, especially at concentration below 1%. While hydrogen peroxide is highly reactive, at pH between 2 and 3, it can be effectively stabilized by various chemicals sequestering the transition metal ions. Below a pH of 2, hydrogen peroxide loses some of its stability. Furthermore, hydrogen peroxide can be concentrated up to 8% without any special packaging or increased transportation costs. Solutions containing more than 8% hydrogen peroxide are classified by the U.S. Department of Transportation (DOT) as an oxidizer. The lower the level of hydrogen peroxide needed in the use concentration, the higher the dilution factor, which leverages the shipping and handling costs. A pH of the diluted teat dip can range from 2.0 to 3.5, though 2.3 to 2.7 were shown to be optimal for a hydrogen peroxide teat dip in terms of the best antimicrobial activity and product stability.

Compositions of the disclosure include at least one germicidal agent. This germicidal agent is preferably hydrogen peroxide in a concentration of between about 0.1% to 30%. In some embodiments, the hydrogen peroxide concentration is less than about 8% by weight of active hydrogen peroxide. In some embodiments, the concentration of hydrogen peroxide in the composition of between about 0.1% and 3%. Other germicidal agents are known in the art and include but are not limited to: organic acids, dodecyl benzene sulfonate, iodine and iodophors, and chlorine based germicidal agents.

Traditional antimicrobial agents are the components of a composition that destroy microorganisms or prevent or inhibit their replication. In some embodiments, the combined organic acid/anionic surfactant(s) antimicrobial embodiments discussed above may be used to replace or eliminate the need for traditional antimicrobial agents in a wide variety of applications. In some embodiments, antimicrobial compositions may be used in combination with these traditional antimicrobial agents, for example, to achieve an effective kill at lower concentrations of traditional antimicrobial agents.

Traditional antimicrobial agents include iodophors, quaternary ammonium compounds, hypochlorite releasing compounds (e.g. alkali hypochlorite, hypochlorous acid), oxidizing compounds (e.g. peracids and hypochlorite), protonated carboxylic acids (e.g. heptanoic, octanoic, nonanoic, decanoic, undecanoic acids), acid anionics (e.g. alkylaryl sulfonic acids, aryl sulfonic acid, alkyl sulfonic acids, alkylaryl sulfuric acid, aryl sulfuric acid, alkyl sulfuric acid, alkylaryl sulfuric acid), chlorine dioxide from alkali chlorite by an acid activator, and bisbiguanides such as chlorhexidine. In some embodiments, the antimicrobial agent is an oxidizing agent. In some embodiments, the antimicrobial agent is hydrogen peroxide, iodine, dodecyl benzene sulfonic acid, quaternary ammonium chlorides, chlorhexidine, sodium hypochlorite, acidified sodium chorite/chlorine dioxide, or combinations thereof. Phenolic antimicrobial agents may be chosen from 2,4,4′-trichloro-2″-hydroxydiphenylether, which is known commercially as triclosan and may be purchased from Ciba Specialty Chemicals as IRGASAN™. and IRGASAN DP 300™. Another such antimicrobial agent is 4-chloro-3,5-dimethyl phenol, which is also known as PCMX and is commercially available as NIPACIDE PX and NIPACIDE PX-P. Other traditional germicides include formaldehyde releasing compounds such as glutaraldehyde and 2-bromo-2-nitro-1,3-propanediol (Bronopol), polyhexamethyl biguanide (CAS 32289-58-0), guanidine salts such as polyhexamethylene guanidine hydrochloride (CAS 57028-96-3), polyhexamethylene guanidine hydrophosphate (89697-78-9), and poly[2-(2-ethoxy)-ethoxyethyl]-guanidinium chloride (CAS 374572-91-5) and mixtures thereof.

In some embodiments, the disclosed germicides may be used in combination with traditional germicides such as copper sulfate, zinc sulfate, sulfamethazine, quaternary ammonium compounds, hydrogen peroxide and/or peracetic acid, for example, to achieve an effective kill at lower concentrations of traditional germicides. In some embodiments, the antimicrobial agent is peracetic acid, hypochlorite, chlorine dioxide, lactic acid, dodecylbenzene sulfonic acid, caprylic acid, salicylic acid, glycolic acid, capric acid, or combinations thereof. In some embodiments, the antimicrobial agent is hydrogen peroxide, iodine, dodecyl benzene sulfonic acid, quaternary ammonium chlorides, chlorhexidine, sodium hypochlorite, acidified sodium chorite/chlorine dioxide, or combinations thereof.

### Buffers and pH

The pH of the solution used in teat treatment is important to achieve the desirable level of microbial kill rate. In this aspect, U.S. Pat. Nos. 4,376,787; and 4,404,040; and U.S. Patent Application No. 2012/0184618 present various sanitizing solutions based on organic acids. All those solutions were found effective in the range of pH 2 to 4, preferably pH 2 to 3. In some embodiments, the pH of the teat dip composition is less than about 7 or between about 2 and 3.5.

U.S. Pat. No. 6,379,685 teaches that organic acids plus oxidant (chlorite) based teat dip blend is effective within the preferable range of pH 2.5 to 3.5. In U.S. Application No. 2013/0089621 the disinfectant solutions containing hydrogen peroxide and amphoteric surfactants displayed satisfactory microbial kill rate in the preferable range of pH 3 to 3.5. In a 2008 paper [S. Raffellini et al, J. Food Safety, 28, 514-533 (2008),] the sanitizing effect of hydrogen peroxide was more specifically studied as a function of pH, although without any involvement of surfactants. It showed systematic enhancement of the killing rate of E. coli at a given hydrogen peroxide concentration, within the range of 0.5% to 3% when pH was reduced from pH 7 to pH 3.

In teat dip applications, contact time may be as short as 30 sec. Effective sanitation within that time period was achieved by reducing pH value of the solution slightly below about pH 3 with the compositions herein. Buffers included are those known in the art and depend on the pH range desired. In some embodiments, the pH is adjusted by methods known to those skilled in the art such as adding phosphoric acid and/or sodium hydroxide.

### Surfactants

A number of different surfactants, or wetting agents, can be used. Surfactants are used to improve the wetting of the surfaces to which they are applied. They reduce the interfacial tension between water and a substrate or other liquid leading to improved wetting. The wetting of a surface significantly increases the contact area on the surface of active ingredients, which facilitates emulsification, solubilization or antimicrobial action to the wetted surfaces. Wetting and penetrating beneath a contaminant on skin, for example, helps to lift off and remove the contaminant and any bacteria that might be harbored by the contaminant.

In some embodiments, the surfactant is selected from the group consisting of an anionic surfactant, a cationic surfactant, a non-ionic surfactant, and an amphoteric surfactant, or a combination thereof. In some embodiments, anionic surfactants are linear and branched, saturated and unsaturated alkyl sulfates, alkyl ether sulfates, alkyl ether phosphates, alkyl carboxylates, alkyl ether carboxylates, and a variety of others known to those skilled in the art. In some embodiments, nonionic surfactants include alkoxylates of alcohols, fatty acids, and esters. In some embodiments, nonionics further include alkyl esters of alkyl fatty acids, alkyl lactates, alkyl lactyl lactates, and alkoxylated sorbitan ester derivatives. Examples of amphoteric surfactants include alkyl dimethyl amine oxides, alkyl amido propyl amine oxides, alkyl dimethyl betaines, alkyl amido propyl dimethyl betaines, alkyl hydroxyl sultaines, and others known to those skilled in the art. In some embodiments, cationic surfactants include esterquats, alkyl trimethyl quaternary ammonium chlorides, alkyl trimethyl quaternary ammonium methyl sulfates, and alkyl pyridinnium chlorides. In some embodiments, the surfactant is selected from those that are approved for food contact by the Food and Drug Administration. In some embodiments, the surfactant is approved for food contact such as those listed in 21 CFR 178.3400.

In some embodiments, the surfactant is selected from group consisting of glycerol, sodium laureth sulfate, ethoxylated phosphate ester, alkyl polyethoxy-propoxy sulfate, linear alcohol ethoxylates, or combinations thereof. In some embodiments, the diluted ready to use form has the concentration of the surfactant in the composition of between about 1% and 40%, 5% and 30%, 10% and 25% or 10% and 20%. In some embodiments, the concentrate has the concentration of the surfactant that is less than 10%, 5%, 2% or 1%.

In some embodiments, surfactants are chosen from those that are gentle and nonirritating to animal skin, including alkyl polyglucosides and sorbitan derivatives. High volume surfactants, such as sulfonates are used extensively, as well. Some of the gentler surfactants are less effective as cleaners, especially when heavier soils are involved. Since the removal of contamination from the bovine skin is the most important item in maintaining good teat health, and subsequently a high milk production rate, the surfactants used are crucial to the teat dip overall performance. This is an especially important feature for the pre-milking procedure, where the bovine skin typically has a higher contamination level than post-milking. The pre-milking cleaning step typically allows only about thirty seconds, which means that the surfactant must provide rapid wetting of the contaminated area. After application of the teat dip, via either dipping or spraying, the teat is wiped off by the dairy operator. In some embodiments, the application of the teat dip composition is performed with dipping or spraying. In some embodiments, following the application of the teat dip composition the teat is wiped off by the dairy operator.

### Emollients

Emollients are a necessary addition to most teat dip compositions. In some embodiments, the emollient may include glycerin, propylene glycol and dipropylene glycol, sorbitol, shea butter, coco butter, allantoin, sorbitol and any number of skin conditioning agents that might also be used for human or animal skin. They act to soothe the teat skin that has been antagonized by repeated chemical exposure from teat dips, manual handling that includes the milking machine, exposure to dirt and related pathogens and environmental exposure including variations of temperature and excessive sun light. One downside to emollients is that they might act as a barrier to the antimicrobial being used in a teat dip. Further, in post-milking application, it is desirable to maintain the antimicrobial agent on the teat surface. However, the emollients are also humectants and absorb moisture, which works against the action of the antimicrobial because a dry teat is less prone to microbial attack. Effective teat dips should not sensitize the teat skin, nor contain unnecessary amounts of emollient.

The addition of emollients to the composition is critical to promoting and maintaining a healthy skin condition by the teat dip composition, compared to antimicrobial action in hard surface cleaning applications. In the compositions of the current disclosure, as well as other commercially available teat dips, the emollients typically comprise the largest single ingredient category in teat dip formulations. In some embodiments, the emollient is glycerol or propylene glycol, wherein the concentration of the emollient in the composition is less than 30%.

Skin conditioning agents may also be optionally used in the disclosed compositions. Skin conditioning agents may provide extra protection for human or animal skin prior to or subsequent to being exposed to adverse conditions. In some embodiments, skin conditioning agents may include moisturizers, such as glycerin, sorbitol, propylene glycol, D-Panthenol, Poly Ethylene Glycol (PEG) 200-10,000, Poly Ethylene Glycol Esters, Acyl Lactylates, Polyquaternium-7, Glycerol Cocoate/Laurate, PEG-7 Glycerol Cocoate, Stearic Acid, Hydrolyzed Silk Peptide, Silk Protein, Aloe Vera Gel, Guar Hydroxypropyltrimonium Chloride, Alkyl Poly Glucoside/Glyceryl Luarate, shea butter and coco butter; sunscreen agents, such as titanium dioxide, zinc oxide, octyl methoxycinnamate (OMC), 4-methylbenzylidene camphor (4-MBC), oxybenzone and homosalate; and itch-relief or numbing agents, such as aloe vera, calamine, mint, menthol, camphor, antihistamines, corticosteroids, benzocaine and paroxamine HCl. In some embodiments, the skin conditioning agent is less than 30% of the composition.

Sequestrants and/or Stabilizers

The effectiveness of a concentrated teat dip has to account for potential poor quality water, including high levels of calcium and magnesium. The teat dip composition of the current disclosure is designed to be compatible with a wide range of water sources, including high levels of calcium and magnesium hardness, but softened or filtered water is always preferred. Methods of improving the performance of products with high levels of calcium and magnesium and known in the art and include using chelants such as EDTA, phosphoric acid, and phosphonic acid derivatives. Sequestrants and/or stabilizers of the present disclosure may be a chelant to include those known in the art such as EDTA, phosphonic acids, etc. In some embodiments, the chelant is based on hydroxyl ethylidene (1,1-diphosphonic acid), noted for its excellent CaCO₃ scale inhibition. In some embodiments, the sequestrant and/or stabilizer is EDTA, a phosphonic acid, hydroxyl ethylidene (1,1-diphosphonic acid), or combinations thereof. In some embodiments, the concentration of the sequestrant and/or stabilizer in the composition is between about 0.1% to 5%.

To reduce the cost of transportation, it is desirable to provide a concentrate which may be diluted by the distributor or at the farm. It is cheaper to store and transport a concentrate as compared to a ready-to-use solution. In some embodiments, the composition is a concentrate that can be diluted to a ready to use form or pre-diluted as the ready to use form. In some embodiments, the concentrate is diluted to the ready to use form with tap water, softened water, filtered water, purified water or combinations thereof.

### Colorants

Colorants are commonly used in teat dip compositions. Coloration is desirable in both concentrate and ready to use form, so that when applied, the milker can be sure of which animals had been treated. It is important that the colors of concentrate and ready to use form are easy to distinguish. Application of colorants in hydrogen peroxide-based teat dips however is limited by the compatibility issues: many organic colorants fade in the presence of hydrogen peroxide, especially at low pH. Only non-toxic and preferably food grade colorants are acceptable in teat dip formulations. Colorants included in the present disclosure are those known in the art such as, but not limited to, Key Acid Food Colorant from Keystone Aniline Corp, IL. Preferred are colorants compatible with peroxides, especially in the pH 2-3 range and approved for food contact. In some embodiments, the dye is a non-toxic dye or a food grade dye.

### Teat Dip Tests

Initially, tests were performed over a 60 day period at two dairies. All samples in the testing were blind labeled. Subsequent use continued for over 10 months, at dairy 1, through all 4 seasons that showed effectiveness in freezing temperatures to 32° F. to extremely hot and dry conditions in excess of 90° F., plus rainy, and snowy conditions. Initial tests in Dairy 1 consisted of 145 Holstein cows, each of which was machine milked, twice daily. Prior to Example 1, an iodine based product was used for both pre- and post-milking. The iodine based product is listed as a 0.5% Iodine pre-milk and 1% Iodine post-milk applications. The level of surfactants and emollients is unknown. Historically, two to three new mastitis cases developed every week with this iodine based teat dip.

Dairy 2 consisted of over 450 Jersey cows, machine milked, twice daily. Prior to Example 1, an iodine based product was used for both pre- and post-milking. The iodine based product is listed as a 0.9% iodine based product. Example 1 was applied as a spray in both pre- and post-milking. Pre-milking application consisted of a 30 second dwell time, with wiping, using single-use towels. Historically, several new mastitis cases developed every week. The post-milking step consisted of spraying and allowing solution to dry in ambient air. Historically, several new mastitis cases developed every week with this teat dip. In the tests, the cows were treated with the agents of the current disclosure, of which the following composition is given here as an example.

In the experiments, the cows were treated with the agents of the current disclosure, of which the following composition is given here as an example.

### EXAMPLES

Example 1 as presented in Table 1 was a ready to use form (RTU) of the teat dip, made by dilution of a concentrate, with potable water (1 volume of concentrate+12 volumes of water) in both pre- and post-milking. The time it took the milkers to perform the pre-milking cleaning step prior to using Example 1 was 30 seconds soaking and thorough wiping with one-time use wipes. The post-milking step consisted of dipping the entire teat and allowing solution to dry.

TABLE 1
Example 1 - RTU FORM OF TEAT DIP - Net Composition

| Raw materials | % | Function |
|---|---|---|
| hydroxyl ethylidene (1,1- | 0.06 | Sequestrant/stabilizer |
| diphosphonic acid) | | |
| Yeast Protein Component | 3.37 | Protein synergist |
| Hydrogen peroxide | 0.41 | Antimicrobial |
| Lactic acid | 0.02 | Antimicrobial and pH buffer |
| Sodium laureth sulfate | 0.54 | Surfactant |
| Alkyl polyethoxy-propoxy | 0.04 | Surfactant |
| sulfate | | |
| Lauryl lactyl lactate | 0.12 | Co-surfactant |
| Propylene glycol | 1.45 | Emollient/Humectant |
| Dipropyleneglycol | 0.29 | Emollient/Humectant |
| 50% NaOH up to pH 3 | <0.1% | pH adjustment |
| water (by dilution and in | 93.7 | Solvent |
| raw materials) | | |

Since the antimicrobial activity is an important indicator of potential value of a teat dip, the RTU form as described above was subjected to the standard antimicrobial test by a certified outside lab using Suspension Time Kill (ASTM E2315) NG4719 method. Two typical microbes known to be related to mastitis in dairy animals were tried in these tests: E. coli, ATCC 8739 (Gram-negative) and S. aureus (Gram-positive), with Tryptic Soy Broth growth medium and target inoculum concentration of 10⁶ CFU/mL. Two contact times were used: 30 seconds and 3 minutes. The inoculation occurred at 36° C. The antimicrobial test results are presented in Tables 2 and 3.

TABLE 2
Example 2 - Bacterial kill: E. coli.
(ASTM E2315) NG4719 Method

| | | | | % Re- | Log Re- |
|---|---|---|---|---|---|
| | | | | duction | duction |
| | | | | Compared | Compared |
| | | | | to Time | to Time |
| Micro- | Time | Test | | Zero | Zero |
| organism | Point | Substance | CFU/mL | Control | Control |
| E. coli | Time | PBS | 1.50E+06 | N/A | |
| ATCC | Zero | Control | | | |
| 8739 | 30 sec | Example 1 | <5.00E+00 | >99.9997% | >5.48 |
| | | RTU TD | | | |
| | 3 min | | 2.50E+01 | 100.00% | 4.78 |

TABLE 3
Example 3 - Bacterial kill: S. aureus.
(ASTM E2315) NG4719 Method

| | | | | % Re- | Log Re- |
|---|---|---|---|---|---|
| | | | | duction | duction |
| | | | | Compared | Compared |
| | | | | to Time | to Time |
| Micro- | Time | Test | | Zero | Zero |
| organism | Point | Substance | CFU/mL | Control | Control |
| S. aureus | Time | PBS | 1.45E+06 | N/A | |
| ATCC | Zero | Control | | | |
| 6538 | 30 sec | Example 1 | <5.00E+00 | >99.9997% | >5.46 |
| | | RTU TD | | | |
| | 3 min | | <5.00E+00 | >99.9997% | >5.46 |

The composition of Example 4 as presented in Table 4 was prepared and resulted in a clear yellow liquid with a pH of 2.3. The composition was then subjected to storage stability tests at 54° C., 4° C., and −20° C. The composition was found to be homogeneous and clear for up to 2 weeks in each case indicating that the product is stable for normal use. Example 4 has the further advantage of using components approved for food contact under 21 CFR 178.3400.

TABLE 4
Example 4 - CONCENTRATE OF ALTERNATE
TEAT DIP FORMULATION

| Raw materials | % | Function |
|---|---|---|
| hydroxyl ethylidene (1,1- | 1.86 | Sequestrant/stabilizer |
| diphosphonic acid) | | |
| Yeast Protein Component | 43.64 | Protein synergist |
| Hydrogen peroxide | 7.95 | Antimicrobial |
| Propylene glycol | 16.73 | Emollient/Humectant |
| Glycerol | 3.63 | Emollient/Humectant |
| Ethoxylated phosphate ester | 7.43 | Surfactant |
| Linear alcohol ethoxylate | 4.01 | Co-surfactant |
| 50% NaOH up to pH 3 | <0.1% | pH adjustment |
| water (in raw materials) | 14.75% | Solvent |

### Field Observations

Determine the effectiveness of the compositions was based on the number of cases of clinical mastitis, as compared to historical results. Clinical mastitis frequency largely determines the productivity of the milking cows for dairy farms.

After the first 24 days of treatment at each dairy, there were zero new cases of mastitis found at either dairy. In each case, the milkers' comments at each dairy were virtually identical. The immediate reaction was that Example 1 was a superior cleaning agent than any they had used before. In the pre-milking cleaning operation, when the bovine teats have the higher level of contamination, the milkers in each dairy observed that the pre-milking contamination was removed very easily and more effectively than compared to any teat dip that had been used in the past.

Dairy 2 found that 3 cows developed mastitis after week six of use of Example 1, two weeks after stopping post-dip due to cold, but above freezing, and wet weather.

It was also noted by milkers at both dairies that for the first few days of using the composition of Example 1 in the pre-milking cleaning step, there was a notable amount of foaming and bubbling. This was believed to be due to the hydrogen peroxide interacting with the high levels of contamination. After several days of using composition of the Example 1, it was noted in both dairies that the level of bubbling and foaming was reduced moderately to substantially, depending on the weather and level of visible soil on the skin surface, and continued to be so in the following weeks. This is believed to be due to the composition of the Example 1 being a more effective cleaning agent, leaving less contamination on the teats surfaces. It was hypothesized that the ability of Example 1 composition to clean not only more quickly, but to remove contamination and penetrating more deeply into the folds and cracks of the skin, was a key factor in eliminating any new mastitis cases during the 30 day period. Though not a limiting factor of the current disclosure, it is believed that improved cleaning can be just as important a feature for an effective teat dip as the disinfecting ability.

The condition of the bovine teat skin was monitored at each dairy. The teat skin showed a decrease in the negative effects of the previously used, harsh teat dip on the teat skin after regular use of the composition of Example 1 Milkers have a keen sensitivity to observing even subtle changes in their cows, in particular the condition of the teats. It was observed that one of the factors to the improved appearance of the teat skin was due to the removal of excess dead skin cells, which had sloughed off. The initial increase in the SCC was believed to be caused by the sloughing off of dead skin cells.

Dead skin cells can harbor bacteria and thus promote development of mastitis cases. Removal of dead skin cells is beneficial in the overall health and productivity of the dairy cows.

In Dairy 1, the use of Example 1 composition was initiated on October 28^{th}, and the somatic cell counts were as follows: 350,000 to 450,000 historical baseline prior to use of Example 1. The weeks immediately prior to introducing Example 1, the SCC was at 350,000. After 5 days of using Example 1, the SCC increased to 390,000. Then after 6 more days of continuous use of Example 1 in both pre-milk and post-milk steps, the SCC dropped to 225,000. On Day 12, the SCC was 220,000. The two days simultaneous testing helped to verify that the unexpected drop in SCC was not a random fluctuation, but a stable trend.

In Dairy 2 the use of Example 1 was initiated on October 29^{th}. In contrast to Dairy 1, which uses a dip method, Dairy 2 sprayed the teat dip in the treatment process. Two days after starting use of Example 1, one cow developed a case of mastitis, but this was attributed to being due to the build-up prior to the use of the Example 1 composition, and not associated with the application of this composition. Though the SCC values did not decrease substantially in November and December of the same year. The values typically increase as the weather gets colder and the SCC values were seen as being very good for the conditions of this particular dairy farm.

Dairy 3 produces raw milk and uses a spray method for applying teat dips. A key factor to note is that Example 1 was used on cows that previously had mastitis. The results of Table 5 compare using chlorine dioxide on cows that had not had mastitis, to using Example 1 on cows that were more susceptible due to previous bouts with mastitis. Once a cow developed mastitis in Dairy 3, it is not used in the production of raw milk. The cows that were treated with Example 1 were therefore cows that had a higher propensity to develop a case of mastitis. Pen No's. 0, 1, 3 and 5 used chlorine dioxide for both pre and post milking. Pen No. 7 used Example 1 for the 6 week test period. In all pens, the cows were retained in pens with saw dust in between milking cycles to reduce the chance of spreading infectious disease.

Results for Dairy 3 indicated that 5.7% of the chlorine dioxide treated raw milk cows had developed mastitis during the 6 week test period. The number of cows that were treated using the composition of Example 1, for both pre and post milking, during the 6 week test period was 5.3% as shown in Table 5. Table 6 defines the number of cows in each respective pen of Table 5. The same observations of improved skin condition were noted by milkers for cows treated with Example 1.

TABLE 5

| Date | Pen # | Lact # | Notes |
|---|---|---|---|
| May 9, 2014 | 0 | 2 | 1st X |
| May 9, 2014 | 0 | 5 | 1st X |
| May 16, 2014 | 0 | 2 | 1st X |
| May 20, 2014 | 0 | 4 | 1st X |
| May 25, 2014 | 0 | 4 | 1st X |
| June 7, 2014 | 0 | 3 | 1st X |
| June 10, 2014 | 0 | 4 | 1st X |
| June 1, 2014 | 1 | 1 | 1st X |
| May 9, 2014 | 3 | 1 | 3rd X mastitis |
| May 22, 2014 | 3 | 1 | 2nd X |
| May 26, 2014 | 3 | 1 | 1st X |
| May 29, 2014 | 3 | 3 | 3rd X mastitis |
| June 19, 2014 | 3 | 1 | Not using freestalls New case |
| May 6, 2014 | 5 | 2 | 3 weeks post fresh |
| May 18, 2014 | 5 | 2 | Freshened with mastitis |
| June 1, 2014 | 5 | 3 | Freshened with mastitis |
| May 11, 2014 | 7 | 2 | 3rd X mastitis |
| May 19, 2014 | 7 | 4 | 4th X |
| May 26 ,2014 | 7 | 5 | 1st X |
| June 4, 2014 | 7 | 2 | 2nd X |

Lact # is defined as the number of lactations.

TABLE 6

| Pen # | Head |
|---|---|
| 0 | 80 |
| 1 | 85 |
| 3 | 70 |
| 5 | 45 |
| 7 | 75 |

The presently disclosed composition and methods are a detailed description of certain specific embodiments of the compositions and methods disclosed herein. For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained. It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. As used herein, the term “comprising” means including elements or steps that are identified following that term, but any such elements or steps are not exhaustive, and an embodiment can include other elements or steps.

The presently disclosed composition and methods are not to be limited in scope by the specific embodiments described herein, which are intended as individual aspects of the presently disclosed composition and methods, and functionally equivalent composition and methods are within the scope of the presently disclosed composition and methods. Indeed, various modifications of the presently disclosed composition and methods, in addition to those described herein will become apparent to those skilled in the art from the foregoing description. Such modifications are intended to fall within the scope of the appended claims.

The following publications are referenced:

1. Kevin C. Kregel. “Heat shock proteins: modifying factors in physiological stress responses and acquired thermotolerance.” (2001) J. Applied Physiol. v. 92(5), pp. 2177-2186.

2. Melchior M B, Vaarkamp H, Fink-Gremmels J. “Biofilms: a role in recurrent mastitis infections?” Vet J. 2006 May; 171(3):398-407.

3. Carme Cucarella,¹ M. Ángeles Tormo,¹ Carles Úbeda,¹ M. Pilar Trotonda,¹ Marta Monzón,² Critòfol Peris,³ Beatriz Amorena,² Íñigo Lasa, and José R. Penadés^{1,4,}* “Role of Biofilm-Associated Protein Bap in the Pathogenesis of Bovine Staphylococcus aureus”, Infect Immun. 2004 April; 72(4): 2177-2185.

4. Dubravka Milanov, S. Lazić, Branka Vidié, Jelena Petrović, D. Bugarski, Zorica \check{S}eguljev. “Slime Production and Biofilm Forming Ability by Staphylococcus Aureus Bovine Mastitis Isolates” Acta Veterinaria (Beograd), Vol 60, No. 2-3, 217-226, 2010: www.doaj.org/doaj?func=openurl&genre=article&issn=05678315&date=2010&volume=60&issue=2-3&spage=217.

5. Michelle Arnold, UK Veterinary Diagnostic Laboratory, and Jeffrey Bewley, Animal and Food Science. “Staphylococcus aureus Mastitis” Copyright ©2011 for materials developed by University of Kentucky Cooperative Extension Programs, University of Kentucky College of Agriculture, Lexington, and Kentucky State University, Frankfort. Issued 10-2011, ID 190 www2.ca.uky.edu/agc/pubs/id/id190/id190.pdf.

6. Jessica Belsito. “Dairy Basics—Herd Health” Progressive Dairyman, 16 Mar. 2012 09:06; www.progressivedairy.com/index.php?option=com_content&id=8334:alternative-teat-dips-weighing-cost-and-quality&Itemid=71; “Alternative teat dips: Weighing cost and quality”.

7. Stephen C. Nickerson, Hill Farm Research Station, Louisiana State University Agricultural Center; Homer, La. “Choosing the Best Teat Dip for Mastitis Control and Milky Quality” Source: NMC-PDPW Milk Quality Conference Proceedings, April 2001, p. 43; www.nmconline.org/articles/teatdip.htm.

8. NMC-PDPW Milk Quality Conference Proceedings, April 2001, pg. 43 www.nmconline.org/articles/teatdip.htm

9. www.americanpharmaceuticalreview.com/Featured-Articles/38885-Antimicrobial-Preservatives-Part-Two-Choosing-a-Preservative/ Antimicrobial Preservatives Part Two: Choosing a Preservative

10. https://books.google.com/books?id=_L1c6rR-Mp4C&pg=PA58&lpg=PA58&dq=antimicrobial+activity+at+acidic+pH&source=bl&ots=ILotvZURQ3&sig=WLtAwGCIBK8tpLwIh3-Z7cAW9Bg&hl=en&sa=X&ei=HveRVPT2KsSyoQSQloL4Cw&ved=0CDYQ6AEwBDgK#v=onepage&q=antimicrobial%20activity%20at%20acidic%20pH&f=false

11. http://www.foodsafetymagazine.com/magazine-archive1/augustseptember-2011/sanitizers-and-disinfectants-the-chemicals-of-prevention/ Food Safety Magazine, August/September 2011

The following US patents and US Patent Applications are also referenced: U.S. Pat. Nos. 8,410,055; 8,153,613; 8,022,037; 7,153,527; 7,109,241; 6,902,747; 6,749,869; 6,630,458; 6,395,289; 6,183,785; 6,030,633; 5,776,469; 5,641,498; 5,534,266; 4,434,181; 4,113,854; 2012/0296940; 2012/0184618; and 2011/0230474.

What is claimed is:

1. A teat dip composition, comprising:
a) a fermentation derived mixture that include low molecular weight protein component;
b) an emollient;
c) one or more surfactants; and
d) an anti-microbial agent.

2. The composition of claim 1, wherein the protein component comprises a fermentation broth recovered from a yeast fermentation process.

3. The composition of claim 1, wherein the protein component comprises a mixture of multiple intracellular proteins, or at least a portion of the mixture including yeast polypeptides obtained from fermenting yeast and yeast heat shock proteins resulting from subjecting a mixture obtained from the yeast fermentation to heat shock.

4. The composition of claim 3, wherein the yeast is selected from the group consisting of Saccharomyces cerevisiae, Kluyveromyces marxianus, Kluyveromyces lactis, Candida utilis, Zygosaccharomyces, Pichia pastoris, and Hansanula polymorpha.

5. The composition of claim 1, wherein the concentration of the protein component in the composition is between about 1% and 60%.

6. The composition of claim 1, further comprising a sequestrant and/or stabilizer, wherein the sequestrant and/or stabilizer is EDTA, a phosphonic acid, or combinations thereof.

7. The composition of claim 6, wherein the concentration of the sequestrant and/or stabilizer in the composition is between about 0.1% and 5%.

8. The composition of claim 1, wherein the surfactant is selected from the group consisting of an anionic surfactant, a cationic surfactant, a non-ionic surfactant, an amphoteric surfactant, or a combination thereof.

9. The composition of claim 1, wherein the surfactant is selected from those that are approved for food contact by the Food and Drug Administration.

10. The composition of claim 1, wherein the surfactant is selected from group consisting of glycerol, sodium laureth sulfate, ethoxylated phosphate ester, alkyl polyethoxy-propoxy sulfate, linear alcohol ethoxylates, or combinations thereof.

11. The composition of claim 1, wherein the concentration of the surfactant in the composition is between about 1% and 40%.

12. The composition of claim 1, further comprising a dye, wherein the dye is a non-toxic dye or a food grade dye.

13. The composition of claim 1, wherein the emollient is glycerol or propylene glycol, wherein the concentration of the emollient in the composition is less than 30%.

14. The composition of claim 1, wherein the antimicrobial agent is an oxidizing agent.

15. The composition of claim 1, wherein the antimicrobial agent is selected from hydrogen peroxide, iodine, dodecyl benzene sulfonic acid, quaternary ammonium chlorides, chlorhexidine, sodium hypochlorite, acidified sodium chorite/chlorine dioxide, or combinations thereof.

16. The composition of claim 1, wherein the antimicrobial agent is selected from peracetic acid, hypochlorite, chlorine dioxide, lactic acid, dodecylbenzene sulfonic acid, caprylic acid, salicylic acid, glycolic acid, capric acid, or combinations thereof.

17. The composition in claim 15, wherein the concentration of the hydrogen peroxide in the composition is between about 0.1% to 30%.

18. The composition in claim 15, wherein the concentration of the hydrogen peroxide in the composition is less than about 8%

19. The composition of claim 1, wherein the composition is a concentrate that can be diluted to a ready to use form or pre-diluted as the ready to use form.

20. The composition of claim 19, wherein the concentrate comprises 1% to 3% hydroxyl ethylidene (1,1-diphosphonic acid), 40% to 50% protein component, 6% to 9% hydrogen peroxide, less than 18% propylene glycol and about 4% linear alcohol ethoxylates.

21. The composition of claim 19, wherein the ready to use form comprises 0.015% to 1% hydroxyl ethylidene (1,1-diphosphonic acid), 1% to 20% protein component, 0.1% to 2% hydrogen peroxide and less than 3% emollient.

22. The composition in claim 19, wherein the concentrate is diluted to the ready to use form with tap water, softened water, filtered water, purified water, or combinations thereof.

23. The composition of claim 1, wherein the pH of the composition is less than about 7.

24. The composition of claim 1, wherein the pH of the composition is between about 2 and 3.5.

25. A method of preventing milk contamination and associated bacterial infections of the teat in dairy animals, the method comprises applying to the teat of the dairy animal the composition of claim 1, wherein the application is effective in treating mastitis of the teat, and wherein the composition is gentle on the teat tissue.

26. The method of claim 25, wherein the protein component comprises a fermentation broth recovered from a yeast fermentation process.

27. The method of claim 25, wherein the protein component comprises a mixture of multiple intracellular proteins, or at least a portion of the mixture including yeast polypeptides obtained from fermenting yeast and yeast heat shock proteins resulting from subjecting a mixture obtained from the yeast fermentation to heat shock.

28. The method of claim 26, wherein the yeast is selected from the group consisting of Saccharomyces cerevisiae, Kluyveromyces marxianus, Kluyveromyces lactis, Candida utilis, Zygosaccharomyces, Pichia pastoris, and Hansanula polymorpha.

29. The method of claim 25, wherein the concentration of the protein component in the composition is between about 1% and 60%.

30. The method of claim 25, wherein the application is performed with dipping or spraying.

31. The method of claim 25, wherein the application of the composition improves the rate of cleaning and contaminant removal.

32. The method of claim 25, wherein the application of the composition removes excess dead skin cells from the teat and udder skin.

33. The method of claim 25, wherein the application of the composition breaks down, removes and prevents biofilms that could harbor mastitis causing bacteria or other pathogens.

34. The method of claim 25, further comprising that the application is performed for both pre and post milking, and with continuous use the number of cases of diary animals with clinical mastitis is reduced.
